(19) United States
(12) Patent Application Publication
(10) Pub. No.: US 2020/0405620 A1
(43) Pub. Date: Dec. 31, 2020

(54) HAIR GROWTH FORMULATION

(71) Applicant: Shalea Gates Dixon, Sacramento, CA (US)

(72) Inventor: Shalea Gates Dixon, Sacramento, CA (US)

(21) Appl. No.: 16/946,559

(22) Filed: Jun. 26, 2020

Related U.S. Application Data
(60) Provisional application No. 62/867,333, filed on Jun. 27, 2019.

(51) Int. Cl. A61K 8/92; A61K 8/67; A61K 8/97; A61Q 7/00

(52) U.S. Cl. CPC A61K 8/922; A61K 8/678; A61K 2800/591; A61Q 7/00; A61K 2800/43; A61K 8/97

(57) ABSTRACT

The present invention provides a hair growth composition, a method of making the composition, and a method of treating a subject to enhance hair growth. The hair growth composition includes about 15 to about 38% by volume Fenugreek seed-infused botanical oil; about 13 to about 23.5% by volume castor oil; and an amount of at least one additional botanical component. The method of making a hair growth composition includes steeping fenugreek seeds in a botanical oil for at least about 9 hours to produce an infused botanical oil; removing the steeped fenugreek seeds from the infused botanical oil; and mixing castor oil, rose oil, peppermint oil, and aloe vera oil with the infused botanical oil. The method of treating a subject includes applying about two drops of a hair growth composition to a scalp every two days and massaging the hair growth composition into the scalp.

### CROSS-REFERENCE TO RELATED APPLICATION

This application claims the benefit of priority of U.S. provisional application number 62/867,333, filed Jun. 27, 2019, the contents of which are herein incorporated by reference.

### BACKGROUND OF THE INVENTION

The present invention relates to hair loss and, more particularly, to a formulation for hair growth.

Hair loss, also known as alopecia or baldness, refers to a loss of hair from part of the head or body. Typically, at least the head is involved. Pattern hair loss primarily affects the top and front of the scalp. In males, the hair loss often presents itself as a receding hairline, while in females, it typically presents as thinning of the hair. Pattern hair loss is believed to be due to genetics.

Current hair regrowth formulas are ineffective. Most hair growth products do not cause hair to grow rapidly, have stimulants, and are not natural based or plant based.

As can be seen, there is a need for an effective formula for hair growth.

The present invention assists hair regrowth after hair loss. This product promotes hair growth by stimulation of the scalp with a combination of natural, plant-based oils. No stimulants are added.

### SUMMARY OF THE INVENTION

In one aspect of the present invention, a hair growth composition is provided, comprising about 15% by volume to about 38% by volume Fenugreek seed-infused botanical oil; about 13% by volume to about 23.5% by volume castor oil; and an amount of at least one additional botanical component sufficient to provide a total of 100% by volume.

In another aspect of the present invention, a method of manufacturing a hair growth composition is provided, comprising steeping fenugreek seeds in a botanical oil for at least about 9 hours to produce an infused botanical oil; removing the steeped fenugreek seeds from the infused botanical oil; and mixing castor oil, rose hip oil, peppermint oil, and aloe vera oil with the infused botanical oil.

In another aspect of the present invention, a method of treating a subject to enhance hair growth is provided, comprising applying about two drops of the hair growth composition to a scalp every two days; and massaging the hair growth composition into the scalp. The hair growth composition comprises castor oil, rose hip oil, peppermint oil, aloe vera oil, and infused botanical oil.

These and other features, aspects and advantages of the present invention will become better understood with reference to the following drawings, description, and claims.

### BRIEF DESCRIPTION OF THE DRAWINGS

FIG. 1 is a schematic view of a formulation according to an embodiment of the invention, illustrating an exemplary combination of ingredients in varying containers;

FIG. 2 is a flow chart of a method of manufacturing the formulation according to an embodiment of the invention;

FIG. 3 is a schematic view of a method of treating a subject according to an embodiment of the invention; and

FIG. 4 is a flow chart of the method thereof.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

As used herein, the phrase “consisting essentially of” refers to a composition of which at least 90% is components discussed herein.

The term “safe and effective amount” as used herein means an amount of a compound or composition such as a topical or system active sufficient to significantly induce a positive benefit, for example, hair growth or regrowth, but low enough to avoid serious side effects, i.e., to provide a reasonable benefit to risk ratio, within the scope of sound judgment of the skilled artisan.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Broadly, one embodiment of the present invention is a formulation that grows hair. The present invention may boost thickness of hair for pattern loss, chemotherapy, alopecia, lupus, and other hair loss causes. The formulation, referred to also as “Tru' Growth”, promotes the regrowth of healthy and strong hair. The present invention is also believed to be successful in treating the scalp of Lupus and thyroid patients.

Preferably, the composition is provided as a gel. The composition may be combined with natural, plant-based carrier adjuvants to produce a serum, a pomade, and a spray, among other products.

The oils of the present invention are preferably obtained by a cold press process. Cold-pressed castor oil is particularly preferred.

Fenugreek-infused botanical oil may be olive oil in which Fenugreek seeds have been steeped. Fenugreek-infused olive oil may be prepared from of about 10% by volume to about 40% by volume fenugreek seeds in olive oil, such as about 19% by volume.

The inventive composition comprises at least one botanical component selected from the group consisting of: aloe vera oil, peppermint oil, fenugreek seed-infused oil, rose hip oil, castor oil, and vitamin E.

The inventive composition may include fenugreek seed-infused oil, castor oil, and at least one botanical component selected from the group consisting of: aloe vera oil, peppermint oil, rose hip oil, castor oil, and vitamin E.

In an embodiment, the composition comprises aloe vera oil, peppermint oil, fenugreek seed-infused oil, rose hip oil, castor oil, and vitamin E.

In certain embodiments, the present invention may include any combination of the following ingredients in the following proportions in percent by volume, up to a total of 100% composition by volume:
Aloe Vera oil about 10-22%
Peppermint oil about 7-14%
Fenugreek-infused olive oil, about 15-38%
Hip Oil about 12-22.5%
Castor oil 13-23.5%
Vitamin E 5-10%

The formulation may be made by mixing the ingredients together at room temperature as follows. Mix castor oil, rose hip oil, peppermint oil, and aloe vera oil. Steep fenugreek seeds in olive oil for about 9 hours to about 36 hours, such as about 18 hours. Mix the olive oil, containing fenugreek seed extract, into the castor oil, rose hip oil, peppermint oil, and aloe vera composition.

In certain embodiments, henna may be mixed with the formula to cover greying hair.

The formulation may be stored in a dropper bottle. Preferably, the invention may be stored in dark bottles to better preserve the formulation.

A method of using the present invention may include applying about two drops every two days to a user's scalp and massaging the formulation into the scalp. The user's hair will generally begin to grow back.

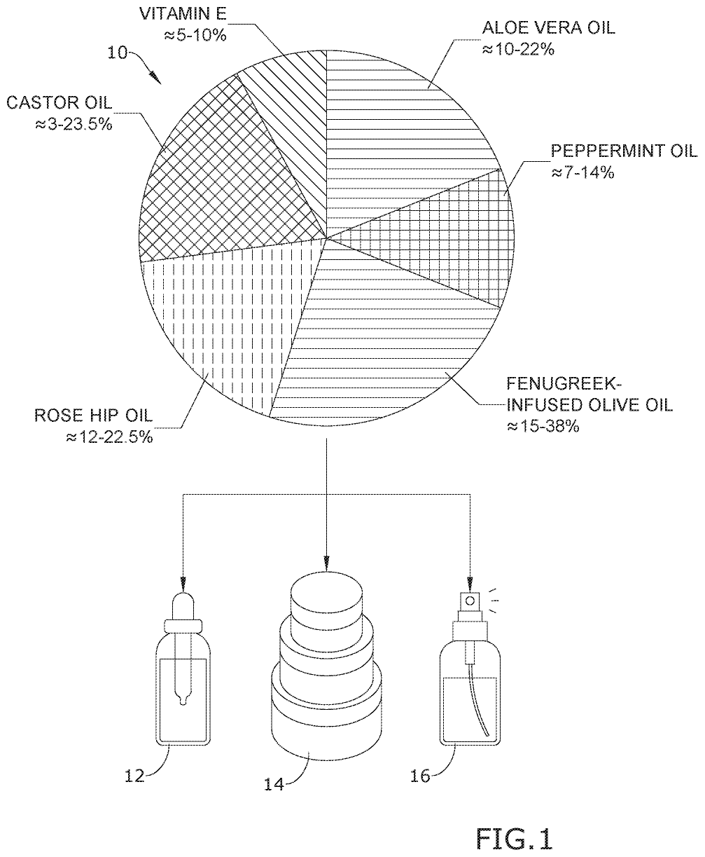

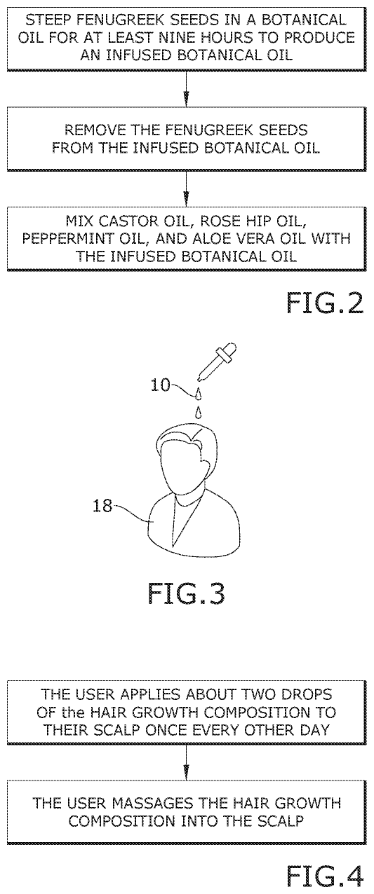

Referring to FIGS. 1 to 4, FIG. 1 illustrates an exemplary plant-based formulation 10 present in a variety of containers, including an exemplary dropper bottle 12, exemplary jars 14, and an exemplary spray bottle 16. The mixing steps for manufacturing the plant-based formulation 10 are shown in FIG. 2. FIG. 3 illustrates the plant-based formulation 10 applied to the head of a user 18 and the process of utilizing the plant-based formulation 10 is shown in FIG. 4.

It should be understood, of course, that the foregoing relates to exemplary embodiments of the invention and that modifications may be made without departing from the spirit and scope of the invention as set forth in the following claims.

What is claimed is:

1. A hair growth composition, comprising:
a. about 15% by volume to about 38% by volume Fenugreek seed-infused botanical oil;
b. about 13% by volume to about 23.5% by volume castor oil; and
c. an amount of at least one additional botanical component sufficient to provide a total of 100% by volume.

2. The hair growth composition of claim 1, wherein the at least one additional botanical component comprises aloe vera oil in an amount of from about 10% by volume to about 22% by volume.

3. The hair growth composition of claim 1, wherein the at least one additional botanical component comprises peppermint oil in an amount of from about 7% by volume to about 14% by volume.

4. The hair growth composition of claim 1, wherein the at least one additional botanical component comprises rose hip oil in an amount of from about 12% by volume to about 22.5% by volume.

5. The hair growth composition of claim 1, wherein the at least one additional botanical component comprises Vitamin E in an amount of from about 5% by volume to about 10% by volume.

6. The hair growth composition of claim 1, wherein the at least one additional botanical component comprises henna in an amount effective to cover greying hair.

7. The hair growth composition of claim 1, wherein the castor oil is cold-pressed.

8. The hair growth composition of claim 1, wherein the at least one additional botanical component comprises:
a. aloe vera oil in an amount of from about 10% by volume to about 22% by volume;
b. peppermint oil in an amount of from about 7% by volume to about 14% by volume;
c. rose hip oil in an amount of from about 12% by volume to about 22.5% by volume; and
d. Vitamin E in an amount of from about 5% by volume to about 10% by volume.

9. A method of manufacturing a hair growth composition, comprising:
a. steeping fenugreek seeds in a botanical oil for at least about 9 hours to produce an infused botanical oil;
b. removing the fenugreek seeds from the infused botanical oil; and
c. mixing castor oil, rose hip oil, peppermint oil, and aloe vera oil with the infused botanical oil.

10. The method of claim 9, further comprising mixing henna into the castor oil, the rose hip oil, the peppermint oil, the aloe vera oil, and the infused botanical oil.

11. A method of treating a subject to enhance hair growth, comprising:
a. applying about two drops of a hair growth composition to a scalp once every other day; and
b. massaging the hair growth composition into the scalp; wherein the hair growth composition comprises castor oil, rose hip oil, peppermint oil, aloe vera oil, and fenugreek-infused botanical oil.
